(19) United States
(12) Patent Application Publication
(10) Pub. No.: US 2026/0188494 A1
(43) Pub. Date: Jul. 2, 2026

(54) METHOD FOR DETERMINING MEDICAL DIAGNOSTIC FACTORS, APPARATUS, DEVICE, AND COMPUTER STORAGE MEDIUM

(71) Applicant: Beijing Yilian Zhishu Technology Co., Ltd., Beijing (CN)

(72) Inventor: Shirui WANG, Beijing (CN)

(73) Assignee: Beijing Yilian Zhishu Technology Co., Ltd., Beijing (CN)

(21) Appl. No.: 19/403,405

(22) Filed: Nov. 28, 2025

(30) Foreign Application Priority Data
Dec. 30, 2024 (CN) 202411980430.4

(51) Int. Cl. G16H 50/20; G16H 10/60; G16H 15/00; G16H 70/60; G16H 80/00

(52) U.S. Cl. CPC G16H 50/20; G16H 10/60; G16H 15/00; G16H 70/60; G16H 80/00

(57) ABSTRACT

Provided is a for determining medical diagnostic factors, including: acquiring an affected area image of a patient, a laboratory report, and a doctor-patient dialogue; performing symptom analysis on the affected area image, the laboratory report, and the doctor-patient dialogue to obtain first diagnostic information corresponding to the affected area image, second diagnostic information corresponding to the laboratory report, and third diagnostic information corresponding to the doctor-patient dialogue; performing a weighted fusion of one or more symptoms to be fused from the first diagnostic information, the second diagnostic information, and the third diagnostic information based on an attention mechanism to obtain target diagnostic information; matching the target diagnostic information with symptoms in a symptom library to determine a target symptom corresponding to the target diagnostic information. The method can efficiently and accurately transform information provided by a patient into consistent medical diagnostic factors.

### CROSS-REFERENCE TO RELATED APPLICATIONS

This application claims priority of Chinese Patent Applicant No. 202411980430.4, filed on Dec. 30, 2024, entitled as “Method for Determining Medical Diagnostic Factors, Apparatus, Device, and Computer Storage Medium,” the entire disclosure of which is incorporated herein by reference for all purposes.

### TECHNICAL FIELD

The present disclosure relates to the field of medical technology, and particularly to a method for determining medical diagnostic factors, an apparatus, a device, and a computer storage medium.

### BACKGROUND

With the development of technology, artificial intelligence technology is being widely applied in the medical field. By means of artificial intelligence technology, valuable medical information can be quickly and accurately extracted.

In the related art, Natural Language Processing (NLP) can be used to obtain effective medical information from doctor-patient dialogues. However, because patients often use non-medical terminology when expressing symptoms, and their descriptions of conditions are subjective and vague, the medical information extracted through natural language processing technology lacks consistency and accuracy. This, in turn, prevents downstream tasks from making predictions based on consistent and accurate medical information, leading to decision-making errors in downstream tasks and causing serious consequences.

Therefore, how to efficiently and accurately transform the information provided by patients into consistent medical information is an urgent technical problem for those skilled in related art.

### SUMMARY

Embodiments of the present disclosure provide a a method for determining medical diagnostic factors, an apparatus, a device, and a computer storage medium, which can efficiently and accurately transform information provided by a patient into consistent medical diagnostic factors.

In a first aspect, an embodiment of the present disclosure provides a method for determining medical diagnostic factors, including: acquiring an affected area image of a patient, a laboratory report, and a doctor-patient dialogue; performing symptom analysis on the affected area image, the laboratory report, and the doctor-patient dialogue through a medical diagnostic factor analysis model to obtain first diagnostic information corresponding to the affected area image, second diagnostic information corresponding to the laboratory report, and third diagnostic information corresponding to the doctor-patient dialogue; performing a weighted fusion of one or more symptoms to be fused from the first diagnostic information, the second diagnostic information, and the third diagnostic information based on an attention mechanism through the medical diagnostic factor analysis model, to obtain target diagnostic information; matching the target diagnostic information with symptoms in a symptom library through the medical diagnostic factor analysis model to determine a target symptom corresponding to the target diagnostic information, where a plurality of symptoms in the symptom library have a hierarchical structure, the hierarchical structure being used to constrain matching priority of the one or more symptoms and determine a symptom context for matching; and matching a corresponding target diagnostic factor from a diagnostic factor library based on the target symptom, where the diagnostic factor library includes diagnostic factors corresponding to the plurality of symptoms.

In a second aspect, an embodiment of the present disclosure provides an apparatus for determining medical diagnostic factors, including:
an acquirer, configured to acquire affected area image of a patient, a laboratory report, and a doctor-patient dialogue;
an analyzer, configured to perform symptom analysis on the affected area image, the laboratory report, and the doctor-patient dialogue through a medical diagnostic factor analysis model to obtain first diagnostic information corresponding to the affected area image, second diagnostic information corresponding to the laboratory report, and third diagnostic information corresponding to the doctor-patient dialogue;
a first determiner, configured to perform a weighted fusion of one or more symptoms to be fused from the first diagnostic information, the second diagnostic information, and the third diagnostic information based on an attention mechanism through the medical diagnostic factor analysis model, to obtain target diagnostic information;
a second determiner, configured to match the target diagnostic information with symptoms in a symptom library through the medical diagnostic factor analysis model to determine a target symptom corresponding to the target diagnostic information, where a plurality of symptoms in the symptom library have a hierarchical structure, the hierarchical structure being used to constrain matching priority of the one or more symptoms and determine a symptom context for matching; and
a third determiner, configured to match a corresponding target diagnostic factor from a diagnostic factor library based on the target symptom, where the diagnostic factor library includes diagnostic factors corresponding to the plurality of symptoms.

In a third aspect, an embodiment of the present disclosure provides a device for determining medical diagnostic factors, including: a processor and a memory storing computer program instructions; where the processor, when executing the computer program instructions, implements the method for determining medical diagnostic factors according to the first aspect or any embodiment of the first aspect.

In a fourth aspect, an embodiment of the present disclosure provides a non-transitory computer-readable storage medium, on which computer program instructions are stored, where the computer program instructions, when executed by a processor, implement the method for determining medical diagnostic factors according to the first aspect or any embodiment of the first aspect.

In a fifth aspect, an embodiment of the present disclosure provides a computer program product, where instructions in the computer program product, when executed by a processor of an electronic device, cause the electronic device to execute the method for determining medical diagnostic factors according to the first aspect or any embodiment of the first aspect.

### BRIEF DESCRIPTION OF DRAWINGS

To more clearly explain the technical solutions in the embodiments of the present disclosure, the accompanying drawings required for the embodiments of the present disclosure will be briefly introduced below. For those of ordinary skill in the art, other drawings can be obtained from these drawings without creative effort.

FIG. 1 is a schematic flowchart of a training method for a dialogue diagnostic analysis model according to an embodiment of the present disclosure;

FIG. 2 is a schematic flowchart of a method for acquiring a fine-tuning dataset according to an embodiment of the present disclosure;

FIG. 3 is a schematic flowchart of a method for acquiring a fine-tuning dataset according to another embodiment of the present disclosure;

FIG. 4 is a schematic flowchart of a method for determining medical diagnostic factors according to an embodiment of the present disclosure;

FIG. 5 is a schematic flowchart of a method for determining medical diagnostic factors according to another embodiment of the present disclosure;

FIG. 6 is a schematic flowchart of a method for determining third diagnostic information according to an embodiment of the present disclosure;

FIG. 7 is a schematic structural diagram of an apparatus for determining medical diagnostic factors according to an embodiment of the present disclosure; and

FIG. 8 is a schematic structural diagram of a device for determining medical diagnostic factors according to an embodiment of the present disclosure.

### DETAILED DESCRIPTION OF THE EMBODIMENTS

The features and exemplary embodiments of various aspects of the present disclosure will be described in detail below. To make the objectives, technical solutions, and advantages of the present disclosure clearer, the present disclosure will be further described in detail below with reference to the accompanying drawings and specific embodiments. It should be understood that the specific embodiments described herein are only for explaining the present disclosure and not for limiting it. For those skilled in the art, the present disclosure can be implemented without some of these specific details. The following description of the embodiments is merely to provide a better understanding of the present disclosure by showing examples of it.

It should be noted that, relational terms such as “first” and “second” are used merely to distinguish one object or operation from another, and do not necessarily require or imply any such actual relationship or order between these objects or operations. Moreover, the terms “includes,” “including,” or any other variation thereof are intended to cover a non-exclusive inclusion, such that a process, method, article, or device that includes a list of elements does not include only those elements but may include other elements not expressly listed or inherent to such process, method, article, or device. Without more constraints, an element preceded by “includes a . . . ” does not preclude the existence of additional identical elements in the process, method, article, or device that includes the element.

With the rapid development of science and technology, artificial intelligence technology is gradually being integrated with the field of medical technology, bringing more intelligent, efficient, and personalized services to the medical industry, which in turn may provide a better user experience for patients.

In related art, medical information provided by patients may exist in various forms. For example, medical information may include doctor-patient dialogues, images of a patient's affected area, and laboratory reports of the patient, among other forms. Furthermore, technologies like NLP can be used to extract effective medical information from the medical information provided by patients. However, traditional NLP technologies often suffer from incomplete information extraction and inaccurate conversion when extracting effective medical information. In addition, because the medical information provided by patients, such as in doctor-patient dialogues, contains non-medical terms and subjective, ambiguous expressions, the effective medical information extracted by NLP often lacks accuracy and consistency. This may lead to risks such as information loss or misjudgment in downstream tasks, like medical clinical decision-making, thereby causing serious consequences.

To solve at least the related technical problems, embodiments of the present disclosure provide a method for determining medical diagnostic factors, an apparatus, a device, and a non-transitory computer storage medium. The method for determining medical diagnostic factors provided by the embodiments of the present disclosure will be introduced first.

It should be noted that, in the determination of medical diagnostic factors provided by the embodiments of the present disclosure, a pre-trained dialogue diagnostic analysis model is needed to extract diagnostic information from doctor-patient dialogues. Therefore, the specific implementation of the training method for the sentiment transfer model and decoding model provided by the embodiments of the present disclosure as described in the drawings will be introduced first.

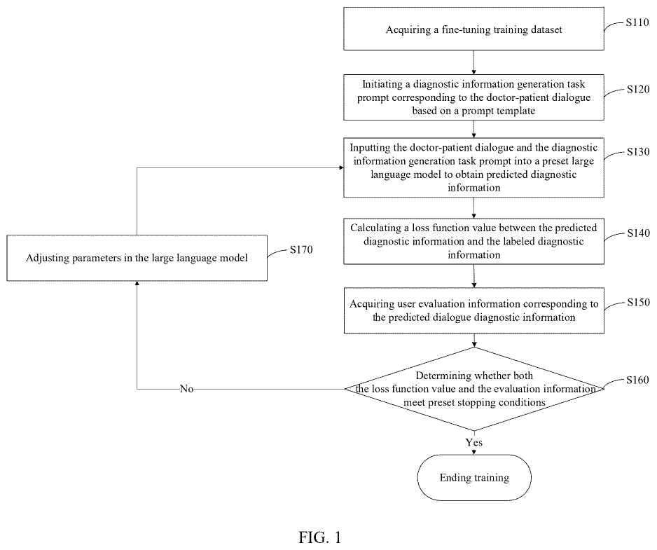

FIG. 1 is a schematic flowchart of a training method for a dialogue diagnostic analysis model according to an embodiment of the present disclosure. As shown in FIG. 1, the training method for the dialogue diagnostic analysis model includes:
S110, acquiring a fine-tuning training dataset.

Wherein, the fine-tuning training dataset includes a plurality of fine-tuning dialogue samples, and each fine-tuning dialogue sample includes dialogue consultation information and corresponding labeled diagnostic information.

In some embodiments, the fine-tuning training dataset may be a training sample dataset used for training the dialogue diagnostic analysis model.

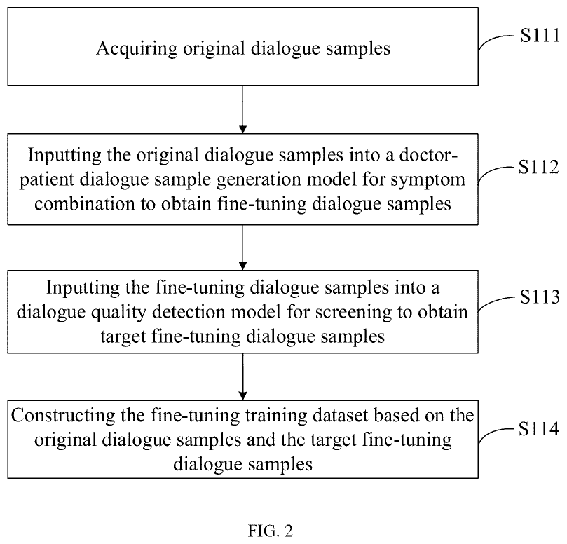

In some embodiments, to improve the accuracy of the dialogue diagnostic analysis model, the fine-tuning training dataset may be a training dataset obtained through sample augmentation techniques. FIG. 2 is a schematic flowchart of a method for acquiring a fine-tuning dataset according to an embodiment of the present disclosure. As shown in FIG. 2, the method for acquiring a fine-tuning dataset includes the following steps:
S111, acquiring original dialogue samples.

In some embodiments, the original dialogue samples may be original dialogue data corresponding to doctor-patient dialogues, where the original dialogue data can be multi-turn dialogue data between a doctor and a patient.

In some embodiments, the original dialogue samples may include original dialogue consultation information and original labeled diagnostic information corresponding to the original dialogue consultation information. Wherein, the original labeled diagnostic information may be annotated according to the hierarchical structure of symptoms in a symptom library.
S112, inputting the original dialogue samples into a doctor-patient dialogue sample generation model for symptom combination to obtain fine-tuning dialogue samples.

In some embodiments, the original dialogue samples can be input into a doctor-patient dialogue sample generation model, and the doctor-patient dialogue generation model performs semantic replacement and symptom combination on the original dialogue samples to obtain fine-tuning dialogue samples.

In some embodiments, based on the labeled diagnostic information in the original dialogue samples, the symptoms corresponding to the original dialogue data can be determined from the symptom library, and the corresponding hierarchical structure of the symptoms can be obtained from the symptom library. Then, the doctor-patient dialogue generation model is used to perform semantic replacement and symptom combination on the original dialogue samples according to the hierarchical structure of the symptoms to obtain fine-tuning dialogue samples.

In some embodiments, fine-tuning dialogue samples can be obtained by combining rule-based generation and deep generation models.

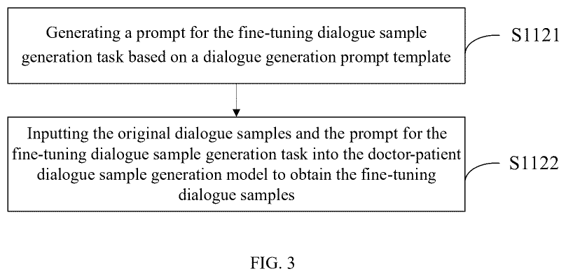

FIG. 3 is a schematic flowchart of a method for acquiring a fine-tuning dataset according to another embodiment of the present disclosure. As shown in FIG. 3, the method for acquiring a fine-tuning dataset includes the following steps:
S1121, generating a prompt for the fine-tuning dialogue sample generation task based on a dialogue generation prompt template.

Wherein, the dialogue generation prompt template includes: multi-turn dialogue data, and a task prompt for generating multi-turn dialogue data layer by layer according to the hierarchical structure of symptoms. Wherein, the symptoms can be symptoms recorded in the symptom library.

In some embodiments, the dialogue generation prompt template can be a prompt template preset by those skilled in the art.

In some embodiments, the prompt templates can be established for various medical topics, where the medical topics can include areas such as cardiovascular, respiratory system, digestive system, etc., to ensure coverage of various types of doctor-patient dialogues. Furthermore, the prompt templates can adopt an inferential prompt structure, which can ensure that the generated dialogue content reflects the changes and trends in the progression of the illness. Wherein, a prompt with an inferential prompt structure can reflect the multi-turn interaction between the doctor and the patient.
S1122, inputting the original dialogue samples and the prompt for the fine-tuning dialogue sample generation task into the doctor-patient dialogue sample generation model to obtain the fine-tuning dialogue samples.

In some embodiments, the original dialogue samples and the prompt for the fine-tuning dialogue sample generation task can be input into the doctor-patient dialogue sample generation model, and the doctor-patient dialogue sample generation model performs semantic replacement and feature combination on the original dialogue samples according to the prompt to obtain fine-tuning dialogue samples.

In the embodiments of the present disclosure, by determining the prompt corresponding to the doctor-patient dialogue sample generation model, the prompt can be used to make the doctor-patient dialogue sample generation model generate fine-tuning dialogue samples. It can be understood that the fine-tuning dialogue samples generated by the doctor-patient dialogue sample generation model can enrich the training data for the dialogue diagnostic analysis model, thereby improving the accuracy of the dialogue diagnostic analysis model. Furthermore, combining the dialogue sample generation model with the symptoms in the symptom library can shorten the collection cycle of actual cases and fill in for scarce scenarios in real life, thereby accelerating the model training speed.
S113, inputting the fine-tuning dialogue samples into a dialogue quality detection model for screening to obtain target fine-tuning dialogue samples.

In some embodiments, the fine-tuning dialogue samples can be screened to ensure that the generated fine-tuning dialogue samples are consistent with actual scenarios, thereby ensuring the data quality of the fine-tuning dialogue samples.

In some embodiments, technical personnel can inspect the fine-tuning dialogue samples to determine that the target fine-tuning dialogue samples conform to actual scenarios.

In some embodiments, the fine-tuning dialogue samples can be input into a dialogue quality detection model, and the dialogue quality detection model screens the fine-tuning dialogue samples to obtain the target fine-tuning dialogue samples. For example, the dialogue quality detection model can score the input fine-tuning dialogue samples and set a score threshold. Only when the score corresponding to a fine-tuning dialogue sample is greater than or equal to the threshold will it be considered a target fine-tuning dialogue sample. This process ensures that only higher-quality, more compliant dialogue samples are selected, thereby improving the accuracy and performance of the dialogue system or model. In addition, the dialogue quality detection model can also provide detailed feedback or suggestions, pointing out potential problems or deficiencies in the fine-tuning dialogue samples, such as unnatural language, logical incoherence, or irrelevant responses.
S114, constructing the fine-tuning training dataset based on the original dialogue samples and the target fine-tuning dialogue samples.

In some embodiments, the fine-tuning training dataset can be constructed based on the original dialogue samples and the target fine-tuning dialogue samples.

In some embodiments, the target fine-tuning dialogue samples can include fine-tuning dialogue consultation information and fine-tuning labeled diagnostic information corresponding to the fine-tuning dialogue consultation information. And, the fine-tuning labeled diagnostic information is annotated according to the hierarchical structure of symptoms in the symptom library. For example, the hierarchical structure of symptoms can include the severity of various symptoms and the location of discomfort, divided according to chronological order.

In the embodiments of the present disclosure, by utilizing the doctor-patient dialogue sample generation model to perform symptom combination on the original dialogue samples, fine-tuned dialogue samples are obtained, thereby achieving data augmentation and increasing the data volume in the samples. Furthermore, the dialogue quality detection model is used to screen the generated fine-tuning dialogue samples, thereby obtaining target fine-tuning dialogue samples that conform to actual scenarios and improving the quality of the samples.
S120, initiating a diagnostic information generation task prompt corresponding to the doctor-patient dialogue based on a prompt template.

In some embodiments, a prompt template can be obtained, and then based on the prompt template, a prompt for the diagnostic information generation task corresponding to the doctor-patient dialogue is generated.
S130, inputting the doctor-patient dialogue and the diagnostic information generation task prompt into a preset large language model to obtain predicted diagnostic information.

Here, the preset large language model (LLM) can perform the task of generating preset dialogue diagnostic information according to the diagnostic information generation task prompt, thereby obtaining the predicted diagnostic information corresponding to the doctor-patient dialogue.

In some embodiments, the predicted diagnostic information can represent information such as the patient's symptoms and signs in the doctor-patient dialogue.
S140, calculating a loss function value between the predicted diagnostic information and the labeled diagnostic information.

The loss function value between the predicted diagnostic information and the labeled diagnostic information can be calculated. In an embodiment, the loss function value between the predicted diagnostic information and the labeled diagnostic information can be determined by calculating the mean squared error or cross-entropy loss between them.
S150, acquiring user evaluation information corresponding to the predicted dialogue diagnostic information.

In some embodiments, Reinforcement Learning from Human Feedback (RLHF) technology can be used to collect feedback from doctors or users on the predicted dialogue diagnostic information as evaluation information.

In an embodiment, the doctor-patient dialogue and the predicted diagnostic information can be displayed to relevant technical personnel, such as multiple doctors, who then evaluate the predicted diagnostic information to obtain multiple pieces of evaluation information. A reward function is then set based on the evaluation information, where the reward function can be based on the score of the evaluation information, or a weighted sum based on metrics such as naturalness and accuracy.
S160, determining whether both the loss function value and the evaluation information meet preset stopping conditions; if yes, ending the training and obtaining the trained dialogue diagnostic analysis model; if no, performing S170.
S170, adjusting parameters in the large language model.

In some embodiments, when it is determined that both the loss function value and the evaluation information meet the preset stopping conditions, the model training can be ended to obtain the trained dialogue diagnostic analysis model.

In some embodiments, when it is determined that either the loss function value or the evaluation information does not meet the preset stopping conditions, step S170 can be performed, which is to adjust the parameters in the preset large language model, and return to initiating the prompt for the diagnostic information generation task corresponding to the doctor-patient dialogue based on the preset prompt template. This allows for the generation of predicted diagnostic information based on the adjusted model, the determination of the evaluation information corresponding to the regenerated diagnostic information, and the calculation of the loss function value corresponding to the regenerated predicted diagnostic information. It is then determined whether the loss function value and evaluation information meet the preset stopping conditions.

In some embodiments, the preset stopping conditions can include the convergence of the loss function or reaching a preset number of iterations, as well as maximizing the expected reward of the reward function or minimizing the expected loss of the reward function.

In the embodiments of the present disclosure, when training the dialogue diagnostic analysis model, Supervised Fine-Tuning (SFT) technology is utilized to train and fine-tune the large language model. This enables the dialogue diagnostic analysis model to more deeply understand the special expressions and corresponding consultation logic within doctor-patient dialogues, thereby accurately extracting the key information in doctor-patient dialogues, i.e., accurate consultation information.

The above is the embodiments of the training method for the dialogue diagnostic analysis model provided in the present disclosure. The dialogue diagnostic analysis model obtained through the above training can be used in the determination of medical diagnostic factors provided in the following embodiments.

The determination of medical diagnostic factors provided in the present disclosure will be introduced below.

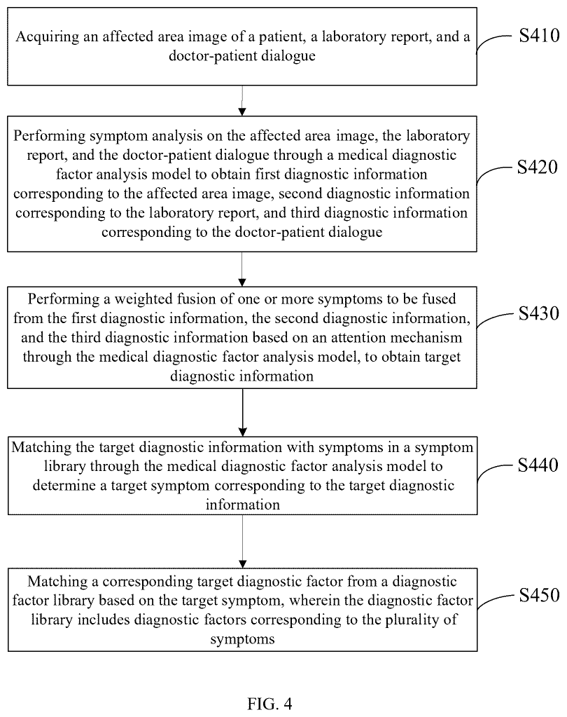

FIG. 4 is a schematic flowchart of a method for determining medical diagnostic factors according to an embodiment of the present disclosure. As shown in FIG. 4, the method for determining medical diagnostic factors includes:
S410, acquiring an affected area image of a patient, a laboratory report, and a doctor-patient dialogue.

In some embodiments, the affected area image, laboratory report, and doctor-patient dialogue can be obtained through multiple data channels, such as by searching for the patient's corresponding case in an electronic medical record system, obtaining the doctor-patient dialogue from real-time doctor-patient chat records, or having the patient or doctor directly upload images or documents.

In some embodiments, the laboratory report can have various forms, such as a laboratory report in a standard format found in an electronic medical record system, or an image of the laboratory report taken by the patient.

In some embodiments, the doctor-patient dialogue can have multiple forms, such as a text-based doctor-patient dialogue or a voice-based doctor-patient dialogue.
S420, performing symptom analysis on the affected area image, the laboratory report, and the doctor-patient dialogue through a medical diagnostic factor analysis model to obtain first diagnostic information corresponding to the affected area image, second diagnostic information corresponding to the laboratory report, and third diagnostic information corresponding to the doctor-patient dialogue.

In some embodiments, the affected area image, the laboratory report, and the doctor-patient dialogue can be input into the medical diagnostic factor analysis model. The model performs symptom analysis on the affected area image, the laboratory report, and the doctor-patient dialogue to respectively obtain the first diagnostic information corresponding to the affected area image, the second diagnostic information corresponding to the laboratory report, and the third diagnostic information corresponding to the doctor-patient dialogue.

In some embodiments, for input data of different dimensions, including the affected area image, the laboratory report, and the doctor-patient dialogue, the medical diagnostic factor analysis model can use different methods to determine their respective corresponding diagnostic information.
S430, performing a weighted fusion of one or more symptoms to be fused from the first diagnostic information, the second diagnostic information, and the third diagnostic information based on an attention mechanism through the medical diagnostic factor analysis model, to obtain target diagnostic information.

In some embodiments, the respective weights of the first diagnostic information, second diagnostic information, and third diagnostic information can be determined based on an attention mechanism. Then, a weighted calculation is performed on the first, second, and third diagnostic information based on their respective weights to obtain the target diagnostic information.
S440, matching the target diagnostic information with symptoms in a symptom library through the medical diagnostic factor analysis model to determine a target symptom corresponding to the target diagnostic information.

Wherein a plurality of symptoms in the symptom library have a hierarchical structure, the hierarchical structure being used to constrain matching priority of the one or more symptoms and determine a symptom context for matching.

In some embodiments, the target diagnostic information can be matched with the symptoms in the symptom library to determine the target symptom corresponding to the target diagnostic information. Here, the target symptom can be a symptom with a hierarchical structure, where the hierarchical structure of the target symptom can be used to represent the symptom context.

In some embodiments, the symptom library can contain various symptoms, signs, and their associated causes. Moreover, the symptoms, signs, and causes are categorized and organized according to a hierarchical structure.

In some embodiments, the matching priority of symptoms can be determined based on the hierarchical structure.

In some embodiments, when hierarchically classifying symptoms, there can be different forms of classification. For instance, when classifying symptoms from coarse to specific, a corresponding symptom tree can be obtained. The target medical diagnostic information can be matched according to the coarse-to-specific hierarchical structure of the symptom tree to obtain the corresponding target symptom. Furthermore, the process of symptom matching can be used as the symptom context.

In an embodiment, the symptom library can be organized into one or more hierarchical structures, for example, by system (such as respiratory system, digestive system, etc.), disease type, or symptom category. Here, each symptom entry may contain standard medical terms, a list of synonyms, a list of dialectal terms, descriptive text, and other metadata. It is understandable that this information helps to improve matching accuracy.

In an embodiment, each symptom entry and its variants in the symptom library can be converted into high-dimensional embedding vectors and stored in an index structure for fast approximate nearest neighbor search. For a given target diagnostic information, the medical diagnostic factor analysis model can first convert it into an embedding vector of the same dimension. The distance between the embedding vector corresponding to the target diagnostic information and the embedding vectors of all symptom entries in the symptom library is calculated. The several closest candidate embedding vectors are selected as potential matches. When the medical diagnostic factor analysis model retrieves matches from the symptom library, it can use the hierarchical structure to impose additional constraints. For example, if a certain symptom is under the subclass of “dyspnea,” other unrelated top-level categories can be excluded. The medical diagnostic factor analysis model may first perform a preliminary screening based on broader categories, and then gradually refine it to more specific subclasses, ensuring that the final matching result is not only based on text similarity but also conforms to logical hierarchical relationships. In addition, the medical diagnostic factor analysis model can also provide a confidence score for each matched symptom to reflect the quality of the match.
S450, matching a corresponding target diagnostic factor from a diagnostic factor library based on the target symptom, wherein the diagnostic factor library includes diagnostic factors corresponding to the plurality of symptoms.

In some embodiments, the diagnostic factor library has a corresponding relationship with the symptom library. By matching the target symptom determined from the symptom library with the various diagnostic factors in the diagnostic factor library, the target diagnostic factor can be obtained.

In some embodiments, the target diagnostic factor can represent key information such as the patient's corresponding symptoms, signs, and examination results.

In the embodiments of the present disclosure, symptom analysis is performed on multi-dimensional medical information of a patient to obtain diagnostic information corresponding to each dimension of medical information. The diagnostic information of different dimensions is then fused based on the model's attention mechanism to obtain the target diagnostic information. Furthermore, by comparing the target diagnostic information with multiple symptoms in the symptom library, the target symptom is determined, and then the diagnostic factor library is used to determine the diagnostic factor corresponding to the target symptom. It is understandable that in the embodiments, by matching the extracted diagnostic information with the symptom library to obtain the target symptom, the target symptom is consistent because it is obtained based on matching with the symptom library. Furthermore, by matching the target symptom with the diagnostic factor library, a standardized diagnostic factor is obtained, providing strong data support for downstream tasks.

In some embodiments, the medical diagnostic factor analysis model can include: an image diagnostic analysis model, a report diagnostic analysis model, and a dialogue diagnostic analysis model. It is understandable that different diagnostic analysis models can be used to process medical information of different dimensions to obtain corresponding diagnostic information.

To determine the respective diagnostic information for the affected area image, the laboratory report, and the doctor-patient dialogue, as another embodiment, the present disclosure further provides another implementation of the method for determining medical diagnostic factors.

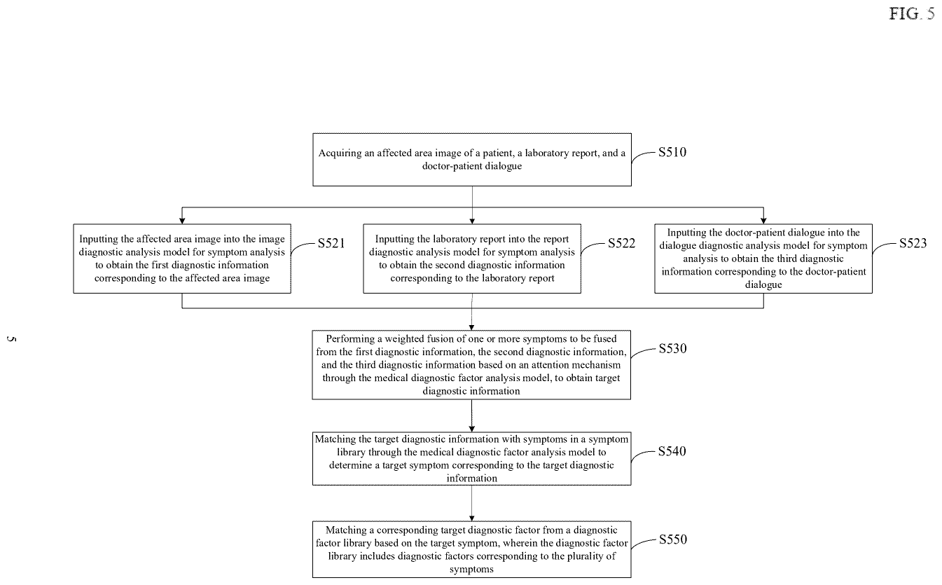

FIG. 5 is a schematic flowchart of a method for determining medical diagnostic factors according to another embodiment of the present disclosure. As shown in FIG. 5, the method for determining medical diagnostic factors includes the following steps:
S510, acquiring an affected area image of a patient, a laboratory report, and a doctor-patient dialogue.
S521, inputting the affected area image into the image diagnostic analysis model for symptom analysis to obtain the first diagnostic information corresponding to the affected area image.
S522, inputting the laboratory report into the report diagnostic analysis model for symptom analysis to obtain the second diagnostic information corresponding to the laboratory report.
S523, inputting the doctor-patient dialogue into the dialogue diagnostic analysis model for symptom analysis to obtain the third diagnostic information corresponding to the doctor-patient dialogue.
S530, performing a weighted fusion of one or more symptoms to be fused from the first diagnostic information, the second diagnostic information, and the third diagnostic information based on an attention mechanism through the medical diagnostic factor analysis model, to obtain target diagnostic information.
S540, matching the target diagnostic information with symptoms in a symptom library through the medical diagnostic factor analysis model to determine a target symptom corresponding to the target diagnostic information.

Wherein a plurality of symptoms in the symptom library have a hierarchical structure, the hierarchical structure being used to constrain matching priority of the one or more symptoms and determine a symptom context for matching.
S550, matching a corresponding target diagnostic factor from a diagnostic factor library based on the target symptom, wherein the diagnostic factor library includes diagnostic factors corresponding to the plurality of symptoms.

In some embodiments, step S510 is consistent with step S410, and steps S530 to S550 are consistent with steps S430 to S450, which will not be described in detail here.

In some embodiments, in S521, the image of the affected area can be input into an image diagnostic analysis model for symptom analysis to obtain the first diagnostic information corresponding to the image of the affected area.

In an embodiment, computer vision technology, such as a convolutional neural network, can be used to analyze the image of the affected area. The convolutional neural network can automatically identify abnormal areas in the image, such as skin lesions, lumps, etc., and extract visual features related to the corresponding areas.

In an embodiment, the convolutional neural network can include an input layer, a convolutional layer, a pooling layer, a fully connected layer, and an output layer. The input layer can be used to receive the preprocessed image data of the affected area. Multiple convolutional layers can be used to extract local features of the affected area image. Each convolutional layer usually contains multiple convolutional kernels, which slide over the input image, perform element-wise multiplication and summation with local regions of the image, and generate new feature maps. A non-linear activation function, such as ReLU, is usually placed after the convolutional layer to introduce non-linearity. After the convolutional layers, pooling layers can be used to downsample the feature maps to reduce the number of parameters and computational load while retaining important features. Common pooling operations include max pooling and average pooling. Furthermore, in the final part of the convolutional neural network, there are usually one or more fully connected layers. These fully connected layers convert the two-dimensional feature maps output by the preceding layers into a one-dimensional feature vector and perform classification or regression tasks. The weight matrix of the fully connected layer performs a matrix multiplication operation with the input feature map to obtain the diagnostic information and outputs the diagnostic information through the output layer.

In some embodiments, in S522, the laboratory report can be input into a report diagnostic analysis model. The report diagnostic analysis model performs symptom analysis on the laboratory report to obtain the second diagnostic information corresponding to the laboratory report.

In some embodiments, the report diagnostic analysis model can adopt Optical Character Recognition (OCR) technology and natural language processing methods to parse the text and numerical information in the report.

In some embodiments, the laboratory report can be preprocessed, including steps like grayscaling and binarization. Layout analysis is performed on the preprocessed laboratory report to determine text areas, image areas, etc., to provide a basis for subsequent character segmentation and recognition. Furthermore, based on the layout analysis, the text in the text areas is segmented into individual characters. This is usually based on features like spacing and connectivity between characters. A natural language model is used to recognize the segmented characters, thereby achieving the parsing of text and numerical values in the report.

In some embodiments, in S523, the doctor-patient dialogue can be input into a dialogue diagnostic analysis model for symptom analysis to obtain the third diagnostic information corresponding to the doctor-patient dialogue. The dialogue diagnostic analysis model is obtained by fine-tuning a large language model with a fine-tuning training dataset.

In some embodiments, by combining supervised fine-tuning and a fine-tuning training dataset, the large language model is fine-tuned to obtain the dialogue diagnostic analysis model.

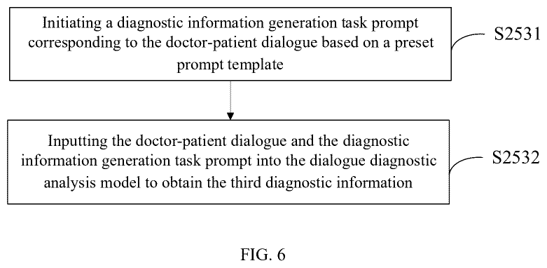

FIG. 6 is a schematic flowchart of a method for determining third diagnostic information according to an embodiment of the present disclosure. As shown in FIG. 6, the method for determining third diagnostic information includes the following steps:
S2531, initiating a diagnostic information generation task prompt corresponding to the doctor-patient dialogue based on a preset prompt template.

Wherein the prompt template includes a hierarchical structure of a plurality of symptoms, and a task prompt for generating diagnostic information layer by layer according to the hierarchical structure

In some embodiments, through the dialogue diagnostic analysis model, the preset prompt template can be obtained, and based on the hierarchical structure of different symptoms in the prompt template, the task prompt for generating diagnostic information can be initiated layer by layer in the order of the hierarchical structure.
S2532, inputting the doctor-patient dialogue and the diagnostic information generation task prompt into the dialogue diagnostic analysis model to obtain the third diagnostic information.

In some embodiments, the dialogue diagnostic analysis model will first understand the content of the doctor-patient dialogue, including the patient's symptom descriptions, medical history information, etc. Furthermore, the dialogue diagnostic analysis model will conduct an in-depth analysis of the dialogue content based on the task prompt, extract key information, and thereby obtain the third diagnostic information corresponding to the doctor-patient dialogue.

In the embodiments of the present disclosure, by combining prompts, the dialogue diagnostic analysis model can be guided to transform non-standardized medical symptoms described by the patient into standard medical symptom terminology, thereby improving the accuracy and consistency of the medical information standardization process.

In some embodiments, by acquiring multi-dimensional medical information and using different diagnostic analysis models for different dimensions of medical information, diagnostic information of different dimensions can be obtained, and then the information corresponding to different dimensions is used to determine the target diagnostic factor. It can be understood that in the embodiments of the present disclosure, the model can receive input data in multiple formats, which enables the model to adapt to different clinical data and improves the applicability of the system.

Based on the method for determining a medical diagnostic factor provided in the above embodiments, the present disclosure further provides a specific implementation of an apparatus for determining a medical diagnostic factor.

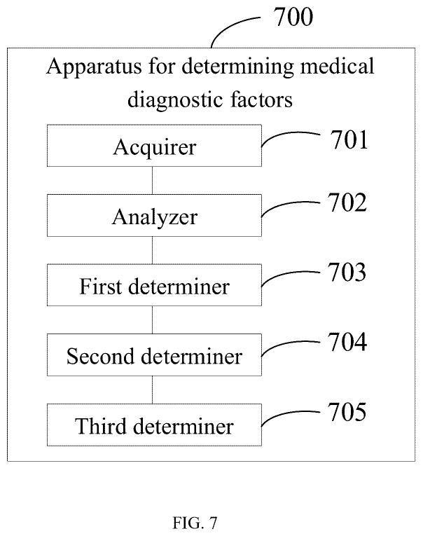

FIG. 7 is a schematic structural diagram of an apparatus for determining medical diagnostic factors according to an embodiment of the present disclosure. As shown in FIG. 7, the apparatus 700 includes:
an acquirer 701, configured to acquire affected area image of a patient, a laboratory report, and a doctor-patient dialogue;
an analyzer 702, configured to perform symptom analysis on the affected area image, the laboratory report, and the doctor-patient dialogue through a medical diagnostic factor analysis model to obtain first diagnostic information corresponding to the affected area image, second diagnostic information corresponding to the laboratory report, and third diagnostic information corresponding to the doctor-patient dialogue;
a first determiner 703, configured to perform a weighted fusion of one or more symptoms to be fused from the first diagnostic information, the second diagnostic information, and the third diagnostic information based on an attention mechanism through the medical diagnostic factor analysis model, to obtain target diagnostic information;
a second determiner 704, configured to match the target diagnostic information with symptoms in a symptom library through the medical diagnostic factor analysis model to determine a target symptom corresponding to the target diagnostic information, wherein a plurality of symptoms in the symptom library have a hierarchical structure, the hierarchical structure being used to constrain matching priority of the one or more symptoms and determine a symptom context for matching; and
a third determiner 705, configured to match a corresponding target diagnostic factor from a diagnostic factor library based on the target symptom, wherein the diagnostic factor library includes diagnostic factors corresponding to the plurality of symptoms.

In some embodiments, the medical diagnostic factor analysis model includes: an image diagnostic analysis model, a report diagnostic analysis model, and a dialogue diagnostic analysis model; the analyzer 702 uses the following method to perform symptom analysis on the affected area image, laboratory report, and doctor-patient dialogue through the medical diagnostic factor analysis model to obtain the first diagnostic information corresponding to the affected area image, the second diagnostic information corresponding to the laboratory report, and the third diagnostic information corresponding to the doctor-patient dialogue: inputting the affected area image into the image diagnostic analysis model for symptom analysis to obtain the first diagnostic information corresponding to the affected area image; inputting the laboratory report into the report diagnostic analysis model for symptom analysis to obtain the second diagnostic information corresponding to the laboratory report; and inputting the doctor-patient dialogue into the dialogue diagnostic analysis model for symptom analysis to obtain the third diagnostic information corresponding to the doctor-patient dialogue, wherein the dialogue diagnostic analysis model is obtained by fine-tuning a large language model with a fine-tuning training dataset.

In some embodiments, the analyzer 702 uses the following method to input the doctor-patient dialogue into the dialogue diagnostic analysis model for symptom analysis to obtain the third diagnostic information corresponding to the doctor-patient dialogue: initiating a diagnostic information generation task prompt corresponding to the doctor-patient dialogue based on a preset prompt template, wherein the prompt template includes a hierarchical structure of a plurality of symptoms, and a task prompt for generating diagnostic information layer by layer according to the hierarchical structure; and inputting the doctor-patient dialogue and the diagnostic information generation task prompt into the dialogue diagnostic analysis model to obtain the third diagnostic information.

In some embodiments, the apparatus further includes a trainer, configured to, before inputting the doctor-patient dialogue into the dialogue diagnostic analysis model for symptom analysis to obtain the third diagnostic information corresponding to the doctor-patient dialogue, acquire a fine-tuning training dataset, wherein the fine-tuning training dataset includes a plurality of fine-tuning dialogue samples, and one or more of the fine-tuning dialogue samples include dialogue consultation information and labeled diagnostic information corresponding to the dialogue consultation information; initiate a diagnostic information generation task prompt corresponding to the doctor-patient dialogue based on a preset prompt template; input the doctor-patient dialogue and the diagnostic information generation task prompt into a preset large language model to obtain predicted diagnostic information, wherein the preset large language model performs a task of generating the predicted dialogue diagnostic information according to the diagnostic information generation task prompt; calculate a loss function value between the predicted diagnostic information and the labeled diagnostic information; acquire user evaluation information corresponding to the predicted dialogue diagnostic information; and in response to determining that either the loss function value or the evaluation information does not meet a preset stopping condition, adjust parameters in the large language model and return to generate the diagnostic information generation task prompt corresponding to the doctor-patient dialogue based on the preset prompt template, until both the loss function value and the evaluation information meet the preset stopping condition, and obtain a trained dialogue diagnostic analysis model.

In some embodiments, the trainer acquires the fine-tuning training dataset in the following manner: acquiring original dialogue samples; inputting the original dialogue samples into a doctor-patient dialogue sample generation model for symptom combination to obtain fine-tuning dialogue samples; inputting the fine-tuning dialogue samples into a dialogue quality detection model for screening to obtain target fine-tuning dialogue samples; and constructing the fine-tuning training dataset based on the original dialogue samples and the target fine-tuning dialogue samples.

In some embodiments, the trainer inputs the original dialogue samples into the doctor-patient dialogue sample generation model for symptom combination to obtain the fine-tuning dialogue samples in the following manner: generating a prompt for the fine-tuning dialogue sample generation task based on a preset dialogue generation prompt template, wherein the dialogue generation prompt template includes: multi-turn dialogue data, and a task prompt for generating multi-turn dialogue data layer by layer according to the hierarchical structure of symptoms; and inputting the original dialogue samples and the prompt for the fine-tuning dialogue sample generation task into the doctor-patient dialogue sample generation model to obtain the fine-tuning dialogue samples.

In some embodiments, the original dialogue samples include original dialogue consultation information and original labeled diagnostic information corresponding to the original dialogue consultation information; the fine-tuning dialogue samples include fine-tuning dialogue consultation information and fine-tuning labeled diagnostic information corresponding to the fine-tuning dialogue consultation information; wherein the original labeled diagnostic information and the fine-tuning labeled diagnostic information are annotated according to the hierarchical structure of symptoms in the symptom library.

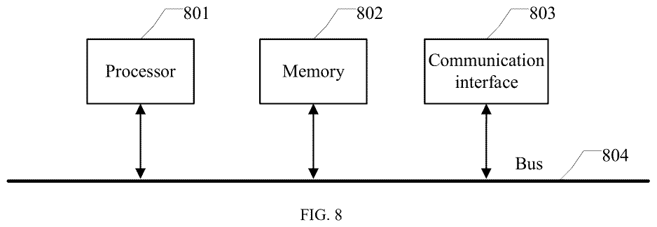

FIG. 8 is a schematic structural diagram of a device for determining medical diagnostic factors according to an embodiment of the present disclosure.

The device for determining medical diagnostic factors may include a processor 801 and a memory 802 storing computer programs or instructions.

In an embodiment, the aforementioned processor 801 may include a Central Processing Unit (CPU), or an Application Specific Integrated Circuit (ASIC), or may be configured to implement one or more integrated circuits of the embodiments of the present disclosure.

The memory 802 may include mass storage for data or instructions. For example, but not limited to, the memory 802 may include a Hard Disk Drive (HDD), a floppy disk drive, flash memory, a compact disk, a magneto-optical disk, a magnetic tape, a Universal Serial Bus (USB) drive, or a combination of two or more of these. Where appropriate, the memory 802 may include removable or non-removable (or fixed) media. Where appropriate, the memory 802 may be internal or external to the integrated gateway disaster recovery device. In a specific embodiment, the memory 802 is a non-volatile solid-state memory.

In an embodiment, the memory may include a read-only memory (ROM), random access memory (RAM), disk storage media devices, optical storage media devices, flash memory devices, electrical, optical, or other physical/tangible memory storage devices. Therefore, generally, the memory includes one or more tangible (non-transitory) computer-readable storage media (e.g., a memory device) encoded with software including computer-executable instructions, and when the software is executed (e.g., by one or more processors), it is operable to perform the operations described with reference to the methods according to the first aspect of the present disclosure.

The processor 801 implements any of the methods for determining medical diagnostic factors in the above embodiments by reading and executing the computer program instructions stored in the memory 802.

In an embodiment, the device for determining medical diagnostic factors may also include a communication interface 803 and a bus 810. As shown in FIG. 8, the processor 801, the memory 802, and the communication interface 803 are connected and communicate with each other via the bus 810.

The communication interface 803 is mainly used to implement communication between the various modules, apparatuses, units, and/or devices in the embodiments of the present disclosure.

The bus 810 includes hardware, software, or both, coupling the components of the device for determining medical diagnostic factors to each other. By way of example and not limitation, the bus may include an Accelerated Graphics Port (AGP) or other graphics bus, an Enhanced Industry Standard Architecture (EISA) bus, a Front-Side Bus (FSB), a HyperTransport (HT) interconnect, an Industry Standard Architecture (ISA) bus, an InfiniBand interconnect, a Low Pin Count (LPC) bus, a memory bus, a Micro Channel Architecture (MCA) bus, a Peripheral Component Interconnect (PCI) bus, a PCI-Express (PCI-X) bus, a Serial Advanced Technology Attachment (SATA) bus, a Video Electronics Standards Association Local (VLB) bus, or other suitable bus, or a combination of two or more of the above. Where appropriate, the bus 810 may include one or more buses. Although the embodiments of the present disclosure describe and illustrate specific buses, the present disclosure may contemplate any suitable bus or interconnect.

The device for determining medical diagnostic factors can execute the method for determining medical diagnostic factors in the embodiments of the present disclosure based on the patient's affected area images, laboratory reports, and doctor-patient dialogues, thereby implementing the method and apparatus for determining medical diagnostic factors described in conjunction with FIG. 4 and FIG. 7.

Additionally, in conjunction with the method for determining medical diagnostic factors in the above embodiments, the embodiments of the present disclosure may provide a non-transitory computer storage medium to implement them. The computer storage medium stores computer program instructions; when the computer program instructions are executed by a processor, they implement any of the methods in the above embodiments.

An embodiment of the present disclosure further provides a computer program product, which includes a computer program. When the computer program is executed by a processor, it implements any method for determining medical diagnostic factors in the above embodiments.

It should be clarified that the present disclosure is not limited to the specific configurations and processes described above and shown in the drawings. For the sake of brevity, detailed descriptions of known methods are omitted here. In the above embodiments, several specific steps are described and shown as examples. However, the method process of the present disclosure is not limited to the specific steps described and shown. Those skilled in the art can make various changes, modifications, and additions, or change the order between steps, after understanding the principle of the present disclosure.

The functional blocks shown in the structural block diagrams described above may be implemented as hardware, software, firmware, or a combination thereof. When implemented in hardware, it can be, for example, an electronic circuit, an application specific integrated circuit (ASIC), appropriate firmware, a plug-in, a function card, and so on. When implemented in software, the elements of the present disclosure are programs or code segments used to perform the required tasks. The program or code segments can be stored in a machine-readable medium or transmitted over a transmission medium or communication link via a data signal carried in a carrier wave. A “machine-readable medium” may include any medium capable of storing or transmitting information. Examples of machine-readable media include electronic circuits, semiconductor memory devices, read-only memory (ROM), flash memory, erasable read only memory (EROM), floppy disks, compact disc read-only memory (CD-ROM), optical disks, hard disks, fiber optic media, radio frequency (RF) links, and so on. The code segments may be downloaded via computer networks such as the Internet, intranets, etc.

It also needs to be stated that the exemplary embodiments mentioned in the present disclosure describe some methods or systems based on a series of steps or devices. However, the present disclosure is not limited to the order of the steps described above, that is, the steps can be executed in the order mentioned in the embodiments, or in a different order from the embodiments, or several steps can be executed simultaneously.

The various aspects of the present disclosure have been described above with reference to flowcharts and/or block diagrams of methods, apparatuses (systems), and computer program products according to embodiments of the present disclosure. It should be understood that each block in the flowcharts and/or block diagrams, and combinations of blocks in the flowcharts and/or block diagrams, can be implemented by computer program instructions. These computer program instructions may be provided to the processor of a general-purpose computer, a special-purpose computer, or other programmable data processing apparatus to produce a machine, such that these instructions, when executed via the processor of the computer or other programmable data processing apparatus, enable the implementation of the functions/actions specified in one or more blocks of the flowchart and/or block diagram. Such a processor may be, but is not limited to, a general-purpose processor, a special-purpose processor, a special application processor, or a field-programmable logic circuit. It can also be understood that each block in the block diagrams and/or flowcharts, and combinations of blocks in the block diagrams and/or flowcharts, can also be implemented by dedicated hardware that performs the specified functions or actions, or can be implemented by a combination of dedicated hardware and computer instructions.

The foregoing are only specific embodiments of the present disclosure. It can be clearly understood by those skilled in the art that, for the convenience and brevity of description, the specific working processes of the systems, modules, and units described above can be referred to the corresponding processes in the foregoing method embodiments, and will not be repeated here. It should be understood that the scope of protection of the present disclosure is not limited to this. Any person skilled in the art can easily conceive of various equivalent modifications or replacements within the technical scope disclosed by the present disclosure, and these modifications or replacements should be covered within the scope of protection of the present disclosure.

What is claimed is:

1. A method for determining medical diagnostic factors, comprising:
acquiring an affected area image of a patient, a laboratory report, and a doctor-patient dialogue;
performing symptom analysis on the affected area image, the laboratory report, and the doctor-patient dialogue through a medical diagnostic factor analysis model to obtain first diagnostic information corresponding to the affected area image, second diagnostic information corresponding to the laboratory report, and third diagnostic information corresponding to the doctor-patient dialogue;
performing a weighted fusion of one or more symptoms to be fused from the first diagnostic information, the second diagnostic information, and the third diagnostic information based on an attention mechanism through the medical diagnostic factor analysis model, to obtain target diagnostic information;
matching the target diagnostic information with symptoms in a symptom library through the medical diagnostic factor analysis model to determine a target symptom corresponding to the target diagnostic information, wherein a plurality of symptoms in the symptom library have a hierarchical structure, the hierarchical structure being used to constrain matching priority of the one or more symptoms and determine a symptom context for matching; and
matching a corresponding target diagnostic factor from a diagnostic factor library based on the target symptom, wherein the diagnostic factor library comprises diagnostic factors corresponding to the plurality of symptoms.

2. The method according to claim 1, wherein the medical diagnostic factor analysis model comprises: an image diagnostic analysis model, a report diagnostic analysis model, and a dialogue diagnostic analysis model;
the performing symptom analysis on the affected area image, the laboratory report, and the doctor-patient dialogue through a medical diagnostic factor analysis model to obtain first diagnostic information corresponding to the affected area image, second diagnostic information corresponding to the laboratory report, and third diagnostic information corresponding to the doctor-patient dialogue comprises:
inputting the affected area image into the image diagnostic analysis model for symptom analysis to obtain the first diagnostic information corresponding to the affected area image;
inputting the laboratory report into the report diagnostic analysis model for symptom analysis to obtain the second diagnostic information corresponding to the laboratory report; and
inputting the doctor-patient dialogue into the dialogue diagnostic analysis model for symptom analysis to obtain the third diagnostic information corresponding to the doctor-patient dialogue, wherein the dialogue diagnostic analysis model is obtained by fine-tuning a large language model with a fine-tuning training dataset.

3. The method according to claim 2, wherein the inputting the doctor-patient dialogue into the dialogue diagnostic analysis model for symptom analysis to obtain the third diagnostic information corresponding to the doctor-patient dialogue comprises:
initiating a diagnostic information generation task prompt corresponding to the doctor-patient dialogue based on a prompt template, wherein the prompt template comprises a hierarchical structure of a plurality of symptoms, and a task prompt for generating diagnostic information layer by layer according to the hierarchical structure; and
inputting the doctor-patient dialogue and the diagnostic information generation task prompt into the dialogue diagnostic analysis model to obtain the third diagnostic information.

4. The method according to claim 2, before the inputting the doctor-patient dialogue into the dialogue diagnostic analysis model for symptom analysis to obtain the third diagnostic information corresponding to the doctor-patient dialogue, further comprising:
acquiring a fine-tuning training dataset, wherein the fine-tuning training dataset comprises a plurality of fine-tuning dialogue samples, and one or more of the fine-tuning dialogue samples comprise dialogue consultation information and labeled diagnostic information corresponding to the dialogue consultation information;
initiating a diagnostic information generation task prompt corresponding to the doctor-patient dialogue based on a prompt template;
inputting the doctor-patient dialogue and the diagnostic information generation task prompt into a preset large language model to obtain predicted diagnostic information, wherein the preset large language model performs a task of generating the predicted dialogue diagnostic information according to the diagnostic information generation task prompt;
calculating a loss function value between the predicted diagnostic information and the labeled diagnostic information;
acquiring user evaluation information corresponding to the predicted dialogue diagnostic information; and
in response to determining that either the loss function value or the evaluation information does not meet a preset stopping condition, adjusting parameters in the large language model and returning to generate the diagnostic information generation task prompt corresponding to the doctor-patient dialogue based on the prompt template, until both the loss function value and the evaluation information meet the preset stopping condition, and obtaining a trained dialogue diagnostic analysis model.

5. The method according to claim 4, wherein the acquiring a fine-tuning training dataset comprises:
acquiring original dialogue samples;
inputting the original dialogue samples into a doctor-patient dialogue sample generation model for symptom combination to obtain fine-tuning dialogue samples;
inputting the fine-tuning dialogue samples into a dialogue quality detection model for screening to obtain target fine-tuning dialogue samples; and
constructing the fine-tuning training dataset based on the original dialogue samples and the target fine-tuning dialogue samples.

6. The method according to claim 5, wherein the inputting the original dialogue samples into a doctor-patient dialogue sample generation model for symptom combination to obtain fine-tuning dialogue samples comprises:
generating a prompt for the fine-tuning dialogue sample generation task based on a dialogue generation prompt template, wherein the dialogue generation prompt template comprises: multi-turn dialogue data, and a task prompt for generating multi-turn dialogue data layer by layer according to the hierarchical structure of symptoms; and
inputting the original dialogue samples and the prompt for the fine-tuning dialogue sample generation task into the doctor-patient dialogue sample generation model to obtain the fine-tuning dialogue samples.

7. The method according to claim 5, wherein the original dialogue samples comprise original dialogue consultation information and original labeled diagnostic information corresponding to the original dialogue consultation information;
the fine-tuning dialogue samples comprise fine-tuning dialogue consultation information and fine-tuning labeled diagnostic information corresponding to the fine-tuning dialogue consultation information;
wherein the original labeled diagnostic information and the fine-tuning labeled diagnostic information are annotated according to the hierarchical structure of symptoms in the symptom library.

8. An apparatus for determining medical diagnostic factors, comprising:
an acquirer, configured to acquire affected area image of a patient, a laboratory report, and a doctor-patient dialogue;
an analyzer, configured to perform symptom analysis on the affected area image, the laboratory report, and the doctor-patient dialogue through a medical diagnostic factor analysis model to obtain first diagnostic information corresponding to the affected area image, second diagnostic information corresponding to the laboratory report, and third diagnostic information corresponding to the doctor-patient dialogue;
a first determiner, configured to perform a weighted fusion of one or more symptoms to be fused from the first diagnostic information, the second diagnostic information, and the third diagnostic information based on an attention mechanism through the medical diagnostic factor analysis model, to obtain target diagnostic information;
a second determiner, configured to match the target diagnostic information with symptoms in a symptom library through the medical diagnostic factor analysis model to determine a target symptom corresponding to the target diagnostic information, wherein a plurality of symptoms in the symptom library have a hierarchical structure, the hierarchical structure being used to constrain matching priority of the one or more symptoms and determine a symptom context for matching; and
a third determiner, configured to match a corresponding target diagnostic factor from a diagnostic factor library based on the target symptom, wherein the diagnostic factor library comprises diagnostic factors corresponding to the plurality of symptoms.

9. A non-transitory computer-readable storage medium, on which computer program instructions are stored, wherein the computer program instructions, when executed by a processor, cause the processor to perform acts comprising:
acquiring an affected area image of a patient, a laboratory report, and a doctor-patient dialogue;
performing symptom analysis on the affected area image, the laboratory report, and the doctor-patient dialogue through a medical diagnostic factor analysis model to obtain first diagnostic information corresponding to the affected area image, second diagnostic information corresponding to the laboratory report, and third diagnostic information corresponding to the doctor-patient dialogue;
performing a weighted fusion of one or more symptoms to be fused from the first diagnostic information, the second diagnostic information, and the third diagnostic information based on an attention mechanism through the medical diagnostic factor analysis model, to obtain target diagnostic information;
matching the target diagnostic information with symptoms in a symptom library through the medical diagnostic factor analysis model to determine a target symptom corresponding to the target diagnostic information, wherein a plurality of symptoms in the symptom library have a hierarchical structure, the hierarchical structure being used to constrain matching priority of the one or more symptoms and determine a symptom context for matching; and
matching a corresponding target diagnostic factor from a diagnostic factor library based on the target symptom, wherein the diagnostic factor library comprises diagnostic factors corresponding to the plurality of symptoms.

10. The storage medium according to claim 9, wherein the medical diagnostic factor analysis model comprises: an image diagnostic analysis model, a report diagnostic analysis model, and a dialogue diagnostic analysis model;
the performing symptom analysis on the affected area image, the laboratory report, and the doctor-patient dialogue through a medical diagnostic factor analysis model to obtain first diagnostic information corresponding to the affected area image, second diagnostic information corresponding to the laboratory report, and third diagnostic information corresponding to the doctor-patient dialogue comprises:
inputting the affected area image into the image diagnostic analysis model for symptom analysis to obtain the first diagnostic information corresponding to the affected area image;
inputting the laboratory report into the report diagnostic analysis model for symptom analysis to obtain the second diagnostic information corresponding to the laboratory report; and
inputting the doctor-patient dialogue into the dialogue diagnostic analysis model for symptom analysis to obtain the third diagnostic information corresponding to the doctor-patient dialogue, wherein the dialogue diagnostic analysis model is obtained by fine-tuning a large language model with a fine-tuning training dataset.

11. The storage medium according to claim 10, wherein the inputting the doctor-patient dialogue into the dialogue diagnostic analysis model for symptom analysis to obtain the third diagnostic information corresponding to the doctor-patient dialogue comprises:
initiating a diagnostic information generation task prompt corresponding to the doctor-patient dialogue based on a prompt template, wherein the prompt template comprises a hierarchical structure of a plurality of symptoms, and a task prompt for generating diagnostic information layer by layer according to the hierarchical structure; and
inputting the doctor-patient dialogue and the diagnostic information generation task prompt into the dialogue diagnostic analysis model to obtain the third diagnostic information.

12. The storage medium according to claim 10, wherein before the inputting the doctor-patient dialogue into the dialogue diagnostic analysis model for symptom analysis to obtain the third diagnostic information corresponding to the doctor-patient dialogue, the processor further performs acts comprising:
acquiring a fine-tuning training dataset, wherein the fine-tuning training dataset comprises a plurality of fine-tuning dialogue samples, and one or more of the fine-tuning dialogue samples comprise dialogue consultation information and labeled diagnostic information corresponding to the dialogue consultation information;
initiating a diagnostic information generation task prompt corresponding to the doctor-patient dialogue based on a prompt template;
inputting the doctor-patient dialogue and the diagnostic information generation task prompt into a preset large language model to obtain predicted diagnostic information, wherein the preset large language model performs a task of generating the predicted dialogue diagnostic information according to the diagnostic information generation task prompt;
calculating a loss function value between the predicted diagnostic information and the labeled diagnostic information;
acquiring user evaluation information corresponding to the predicted dialogue diagnostic information; and
in response to determining that either the loss function value or the evaluation information does not meet a preset stopping condition, adjusting parameters in the large language model and returning to generate the diagnostic information generation task prompt corresponding to the doctor-patient dialogue based on the prompt template, until both the loss function value and the evaluation information meet the preset stopping condition, and obtaining a trained dialogue diagnostic analysis model.

13. The storage medium according to claim 12, wherein the acquiring a fine-tuning training dataset comprises:
acquiring original dialogue samples;
inputting the original dialogue samples into a doctor-patient dialogue sample generation model for symptom combination to obtain fine-tuning dialogue samples;
inputting the fine-tuning dialogue samples into a dialogue quality detection model for screening to obtain target fine-tuning dialogue samples; and
constructing the fine-tuning training dataset based on the original dialogue samples and the target fine-tuning dialogue samples.

14. The storage medium according to claim 13, wherein the inputting the original dialogue samples into a doctor-patient dialogue sample generation model for symptom combination to obtain fine-tuning dialogue samples comprises:
generating a prompt for the fine-tuning dialogue sample generation task based on a dialogue generation prompt template, wherein the dialogue generation prompt template comprises: multi-turn dialogue data, and a task prompt for generating multi-turn dialogue data layer by layer according to the hierarchical structure of symptoms; and
inputting the original dialogue samples and the prompt for the fine-tuning dialogue sample generation task into the doctor-patient dialogue sample generation model to obtain the fine-tuning dialogue samples.

15. The method according to claim 13, wherein the original dialogue samples comprise original dialogue consultation information and original labeled diagnostic information corresponding to the original dialogue consultation information;
the fine-tuning dialogue samples comprise fine-tuning dialogue consultation information and fine-tuning labeled diagnostic information corresponding to the fine-tuning dialogue consultation information;
wherein the original labeled diagnostic information and the fine-tuning labeled diagnostic information are annotated according to the hierarchical structure of symptoms in the symptom library.
